(19) United States
(12) Patent Application Publication
(10) Pub. No.: US 2021/0398625 A1
(43) Pub. Date: Dec. 23, 2021

(54) CASE-LEVEL REVIEW TOOL FOR PHYSICIANS

(71) Applicant: Maximus, Inc., Austin, TX (US)

(72) Inventors: Mark E. Erard, Reston, VA (US); Raja Balasubramanian, Austin, TX (US); Viraf Bankwalla, Austin, TX (US); Michael C. Monette, Austin, TX (US); Mohammad A. Sabri, Austin, TX (US); Bruce W. Saunders, Austin, TX (US); Thomas Stockton, Austin, TX (US); Ryan Vaniderstine, Austin, TX (US)

(21) Appl. No.: 17/358,399

(22) Filed: Jun. 25, 2021

Related U.S. Application Data
(63) Continuation of application No. 15/430,142, filed on Feb. 10, 2017, now abandoned.

(51) Int. Cl. G16H 10/60

(52) U.S. Cl. CPC G16H 10/60; G06Q 2220/00

(57) ABSTRACT

A method of implementing a secure exchange portal system for independent medical review, and classification tools and case-level physician review tools for use in such reviews, are disclosed.

### BACKGROUND

### Field

The present invention relates to the field of services and systems that are used to administer and resolve claims. Embodiments of the invention relate to automation of secure exchange of electronic documents over a network related to independent medical review of worker's compensation claims.

### Description of the Related Art

Government and business entities that are involved in administering and resolving claims currently need to gather, sort, and/or identify large amounts of data from a variety of different sources. Once all of the necessary data is acquired it must be made available to a variety of different entities and persons with various different interests and goals. Sometimes the goals of different entities requiring access to the data are in opposition to one another. At least some of the data may be confidential and/or privileged and as such only certain groups may be allowed access to certain portions of the data.

One such example of administering and resolving claims includes independent medical review of worker's compensation claims in order to provide claims determinations for health insurance payers, workers compensation insurance payers or disability insurance payers. An Independent Medical Review (IMR) process is one in which medical doctors, relying on evidence-based treatment guidelines, have replaced judges as the first level arbiters in resolving disputes over the necessity of recommended medical services. These medical doctors are contracted by an independent review organization, to provide objective, unbiased determinations on what the root cause of the treatment was, whether there is medical necessity, if there was a sentinel event, what was the reason for it, etc.

The IMR process is one which requires the review of numerous different files from different agencies (e.g., hospitals, doctors, insurance agencies, government agencies, police departments, fire departments, emergency services, attorneys, etc.) by numerous different groups (e.g., doctors, attorneys, insurance agents, etc.). These files and/or reports for even just a single case can number in the thousands of pages of documents.

As such, electronic files of such documents can be very large (several hundred pages) sometimes taking a long time to download. Physicians spend significant time searching these large documents for just a handful of useful forms. Attorneys and injured workers may resubmit the same documents since they are unsure what information was provided by the claims administrators. These duplicate submissions results in duplicate work—quite often performed by high-cost physicians.

Also, in files compiled from scanning documents from various sources at various times, documents in the file are very likely to be presented unordered, making it difficult to locate the relevant recent few documents that a physician needs to begin the review. In addition, many documents received in the system are scanned images of paper forms. The images cannot support text searches for key terms like a specific drug or medical condition.

### SUMMARY

Systems and methods of providing secure exchange of electronic documents over a network related to independent medical review of worker's compensation claims, including a secure document exchange portal. Document classification tools and case-level physician review tools, which can be used in conjunction with a secure document exchange portal, are also disclosed.

In an embodiment, a secure exchange portal system for medical review includes a receiving system. The receiving system first authenticates and then authorizes the user to submit information. The system offers several alternative methods to provide files including web form uploads and web service based submissions. Regardless of which method is employed, the receiving system divides the incoming documents into individual smaller chunks, encrypts each of the chunks, stores them and creates metadata. By subdividing documents into smaller components, the secure exchange portal system can be used by stakeholders to efficiently access select portions of documents of the case, without having to download or access entire document files. The system controls access to each document depending on the authorizations of the user.

In an embodiment, an automated document classification system for files under medical review includes a classification system implemented on one or more computer systems. A classification mechanism uses an ensemble of classifiers to identify specific information in files (e.g., a request for authorization, a utilization review determination, or a physician's progress report).

In an embodiment, a physician's case-level review tool includes a search mechanism and leverages meta-data from the automated document classification system. The physician search tool provides one or more files having scanned medical records that are associated with a case under review. The file(s) include information classified by the classification mechanism as a pre-defined information type (e.g., a request for authorization, a utilization review determination, or a physician's progress report). A selection of one or more elements of classified information is received from a physician user. One or more search terms are received from the physician user. The search mechanism performs a search based on the search terms. At least a portion of at least one of the elements of classified information is displayed to the physician user based on the search.

In some embodiments, a method of secure exchange of information for medical review includes receiving a data stream from at least a first source, wherein the data stream is associated with an independent medical review. In some embodiments, the method may include dividing the received data stream into a plurality of chunks. The method may include encrypting the plurality of chunks.

In certain embodiments, a method of managing a secure exchange portal system includes creating metadata for each of the stored documents. The metadata may include a block size and an initialization vector. The method may include using metadata to restrict access to some documents in a case to a first group of users. In some embodiments, the document metadata may also be used to restrict access to some case documents to a second set of users. In some embodiments, documents within a case may be restricted from users in both groups, accessible by both groups or accessible by members of one group and restricted to members of the other.

In some embodiments, the first source comprises at least one paper document converted into an electronic file format. In some embodiments, the metadata comprises coded medical identifiers and/or an encryption key.

In some embodiments, the method includes administering claims for a worker's compensation claim using the plurality of chunks. The method may include resolving claims for a worker's compensation claim using the plurality of chunks.

In some embodiments, at least one of the plurality of chunks comprises multiple scanned documents. The method may include converting the data stream into a searchable format using character recognition. The method may include compressing the plurality of chunks.

In some embodiments, the method includes providing a first access portal for a first group and a second access portal for a second group, wherein the first group and the second group are different.

In some embodiments, users from multiple groups may be able to access the system through the same portal. In such a system, individuals of each group would have access afforded to them appropriate to the group in which they reside.

### BRIEF DESCRIPTION OF THE DRAWINGS

FIG. 1 is a flowchart illustrating a method of managing a secure exchange portal system for independent medical review.

FIG. 2 illustrates one embodiment of a system for managing a secure exchange portal system for independent medical review.

FIG. 3 is a block diagram of one embodiment of a secure exchange portal system.

FIG. 4 illustrates a display of a home page for a user of a secure document exchange portal.

FIG. 5 illustrates a classification tool according to one embodiment.

FIGS. 6A-B illustrate one embodiment of a flowchart illustrating a method of using rules for classifying information in a large electronic file.

FIG. 7 is a flowchart illustrating a method of assisting physicians using a physician's search tool based on information that has been automatically classified.

FIG. 8 is a flowchart illustrating a method of assisting physicians using a physician's search tool.

FIG. 9 illustrates one embodiment of a system including a physician's search tool.

FIG. 10 illustrates a case document management display for a physician search tool according to one embodiment.

FIG. 11 illustrates a display of physician search tool with a Bookmarks tab selected and bookmark links arranged by type.

FIG. 12 illustrates a display of a physician search tool with a Bookmarks tab selected and bookmark links arranged by page.

FIG. 13 illustrates a display of a physician search tool with a Notes tab selected and stick notes attached to a displayed document.

FIG. 14 illustrates a display of a physician search tool with line-by-line search results shown next to a document classified by the system as a Progress Note.

While the invention is described herein by way of example for several embodiments and illustrative drawings, those skilled in the art will recognize that the invention is not limited to the embodiments or drawings described. It should be understood, that the drawings and detailed description thereto are not intended to limit the invention to the particular form disclosed, but on the contrary, the intention is to cover all modifications, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including, but not limited to.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task. In some contexts, “configured to” may be a broad recitation of structure generally meaning “having a feature that” performs the task or tasks during operation. As such, the component can be configured to perform the task even when the component is not currently on.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. Regarding the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for describing embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION OF ILLUSTRATIVE EMBODIMENTS

### Definitions

Unless defined otherwise, all technical and scientific terms used herein have the same meaning as commonly understood by one of ordinary skill in the art.

The term “chunk” as used herein generally refers to a portion of a data stream or file which is sent to a processor or one of the parts of a computer for processing. An example of a chunk may be a portion of a file including a set of scanned documents. The documents may be associated with a case that is under medical review.

“Information” may be in the form of content contained within, or derived from, a file or stream of data. An element or piece of “information” may be a particular scanned document or an element within a scanned document, such as a physician's progress report. “Information” may, in some cases, be in the form of other discrete items appearing within a document or file, such as an attorney letter, or a reproduction of an x-ray image. As used herein, “classified information” includes information that has been classified (e.g., into one or more categories). Information may be classified, for example, using automatic classification mechanisms as described herein.

The term “back-end system” as used herein generally refers to computer systems deal with databases and data processing components, so the purpose of the back-end system is to respond to front-end system requests and operations.

### EMBODIMENTS

### 1. Secure Document Exchange Portal System with Efficient User Access

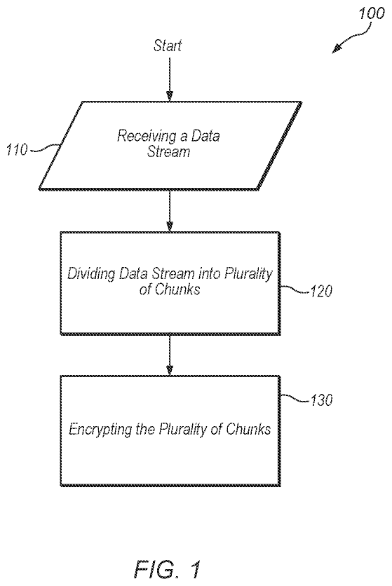

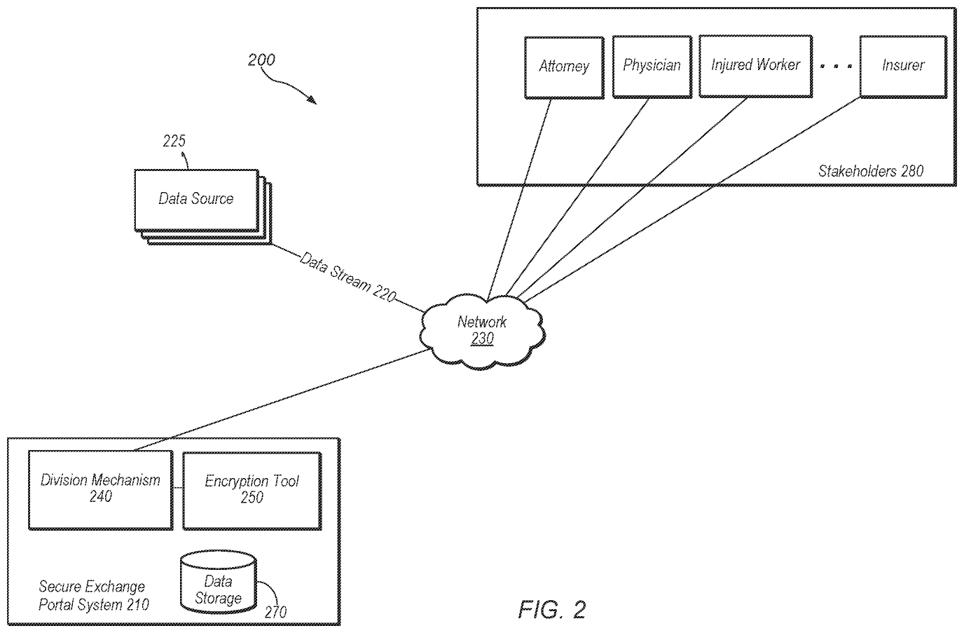

A method may include managing a secure exchange portal system for independent medical review. FIGS. 1-2 illustrate one embodiment of a flowchart illustrating a method (100) and system 200 of managing a secure exchange portal system 210 for independent medical review. The method may include receiving (110) a data stream 220 from one or more sources 225. In some embodiments, the data stream is associated with an independent medical review of a medical claim (e.g., a worker's compensation claim). A document exchange portal system 210 may provide secure exchange of electronic information over a network 230. The system may be used to provide for exposure and consumption of information for administering and resolving claims, such as independent medical review of worker's compensation claims.

The system includes a mechanism that stores, manages and enables review of very large files and facilitates secure access to specific portions or aspects of case information by interested stakeholders. In some embodiments, the method may include dividing (120) the received data stream into a plurality of chunks using a division mechanism 240. In some embodiments, the method may include encrypting (130) the plurality of chunks using an encryption tool 250. In some embodiments, the first source may each include at least one or more paper documents converted into an electronic file format. In some embodiments, at least one of the plurality of chunks comprises multiple scanned pages. The method may include converting the data stream into a searchable format using character recognition. The method may include compressing the individual chunks.

The system may administer and control the rights/access that various stakeholders 280 (e.g., physicians, IMR review staff, injured workers, insurer personnel, and attorneys) have to contribute (e.g., upload) and/or access particular information (e.g., medical records). For example, a reviewing physician may have the right to access only the medical records for those claims that the physician is assigned to review. Individuals and/or groups may be provided with means for accessing encrypted individual chunks. In some embodiments, the method may include establishing a user as a representative of an authorized party thereby providing the user with a defined set of capabilities with regard to particular information (e.g., medical records and access thereof).

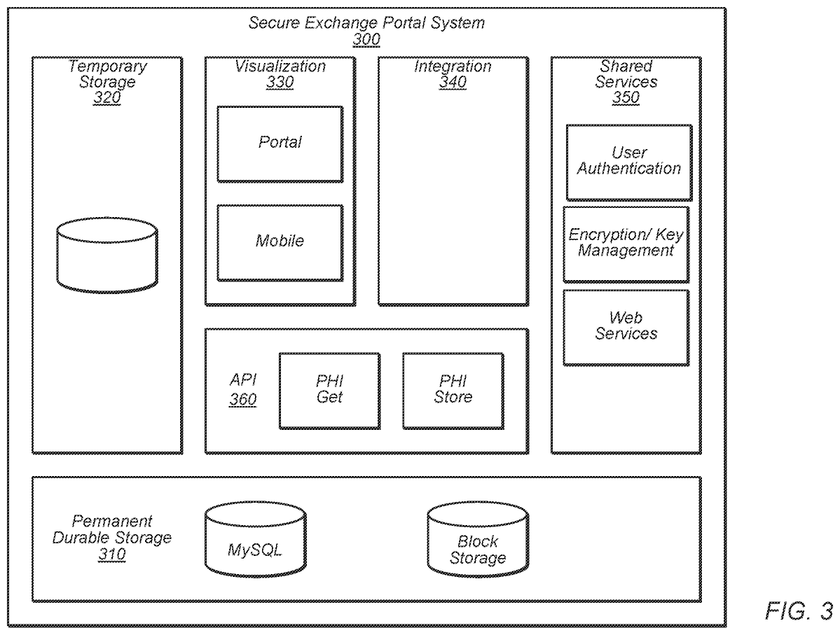

FIG. 3 is a block diagram of one embodiment of a secure exchange portal system. Secure exchange system 300 includes permanent durable storage 310, temporary storage 320, visualization elements 330, integration elements 340, shared services 350, and application program interface 360.

In one embodiment, an integration server manages and controls the exchange of information between one or more back-end servers and a front-end system. The integration server may ensure that any stakeholder access to information on the back-end server is accomplished by secure exchange. In one embodiment, integration services provide a secure gateway to case and document data. The integration server performs message routing and transformation, protocol translation, and request queuing and retries between the different backend services in a secure way. The integration server may provide notification to the back-end system when a new case is received, when documents are received in a mailroom, and when documents are uploaded through portal.

In certain embodiments, metadata is stored through a mechanism that uses CNRI's Digital Object Architecture. A digital object database serves as an information store in response to information retrieved and sent from permanent storage to users. The DOA-based mechanism stores metadata (which may include information about the cases and documents). The mechanism helps facilitate who has what rights to those cases and documents. Using the system, a large number and volume of external users can be supported in separate realms. Each stakeholder may invite other users to receive access according to parameters set by the inviting stakeholder and the system. For example, a claimant may invite the claimant's attorney to allow the attorney to upload and/or access documents.

A set of web servers are responsible for serving up portal pages and access the web services behind those pages.

Secure exchange system 300 may include document upload and retrieval tools. Document upload tools allow both individual and batches of documents to be transferred to the portal and automatically assigned to cases as needed. In some embodiments, a Retrieval tool and PDF viewer enables a user to retrieve the content required to render/view a single page vs. retrieving the entire file, thereby saving download time. The retrieval tool may be configured to restrict actions available to a user—for example, printing of the PDF file may be disabled, as can the ability to save the document to local storage.

Data is preferably encrypted at rest and in transit. Access control may be bound to each object (medical record). Encryption keys may be stored securely using a key management tool. Multiple sources of encryption keys may be used and stored independently as metadata such that an unauthorized user is unable to assemble sufficient keys in order to decrypt the data. Data services are provided such that documents cannot be decrypted without first establishing permissions via authentication and authorization.

In some embodiments, the system retrieves a list of documents for a specified case. The document list includes key metadata including document unique handle id, created date, last updated date, document source, and document type. A specific document may be retrieved based on its unique handle id. A list of active cases for the Claims Administrator may be retrieved. Case metadata may include case handle id, current case status, case status date, date case last updated.

In some embodiments, a secure document exchange system includes an archiving mechanism.

In some embodiments, the system may handle authentication of each user depending on the role of the user. The method may include using a first set of metadata to restrict access to at least one of the documents to a first group of users and a second set of metadata to restrict access to at least one of the documents to a second group of users. The first group of users may be different from the second group of users. The first group of users may access a specific portion of the at least one of the documents using the metadata.

In some embodiments, security/access may be handled by an access management tool. In some embodiments, an open source tool may be used as the access management tool. The access management tool may include one or more features. In some embodiments, an access management tool may allow for single-sign on. Single-sign on may allow for authentication and signing in to multiple different platforms with a single login/logout. In some embodiments, an access management tool may allow for login via social networks via configuring the Identity Provider through the admin console. In some embodiments, an access management tool may allow for connecting through Active

Directory servers. In some embodiments, an access management tool may allow for central management of all of the accessibility features. In some embodiments, an access management tool may allow for enabling and disabling various features (e.g., configuring identity brokering and user federation).

In some embodiments, the method includes administering claims for a worker's compensation claim using the plurality of chunks. The method may include resolving claims for a worker's compensation claim using the plurality of chunks. In some embodiments, the metadata associated with chunks and/or documents may include coded medical identifiers. Coded medical identifiers may include, for example, industry standardized medical labels or identifiers.

The system may provide access to information in the documents to each of the many stakeholders who may be involved in a particular case (e.g., an appeal of a worker's comp claim). The system may provide access at high volume to a large number of documents in a secure manner. The system may enable a stakeholder to locate and gain access to a specific desired portion of a record (for example, page 432 of a 500 page file containing hundreds of records) without loading and transferring an extremely large file.

In some embodiments, the method includes providing a first access portal for a first group and a second access portal for a second group, wherein the first group and the second group are different.

In some embodiments, the system may allow participants using the system to request or administer secure access by related persons based on the related person's role. For example, an injured worker may be able to initiate a request for and/or enable limited access for the injured worker's attorney. The injured worker's ability to enable access to the injured worker's attorney may be limited by the injured workers own limited access. The system may provide a window to accessing or providing information that is restricted based on the related person's role. For example, an injured worker's attorney may be only able to view, download, or upload documents for those claims where the attorney is representing the injured worker. The operation of the mechanism may vary depending on the type of stakeholder. For example, a large claims administrator may have one manner of enabling access by others (e.g., claims handling by numerous staff members at an insurer) and an individual injured worker may have a different manner of enabling access.

In some embodiments, a secure exchange portal system for independent medical review may include a receiving system implemented on one or more computer systems. The receiving system may be configured to implement a method as described herein.

In some embodiments, a non-transitory, computer-readable storage medium including program instructions stored thereon. The program instructions, when executed on one or more computers, may cause the one or more computers to implement a receiving system configured to implement a method as described herein.

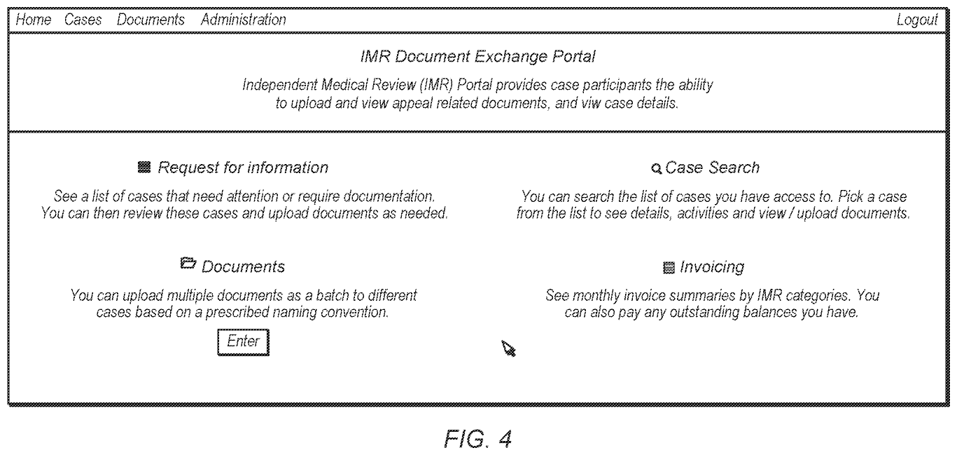

FIG. 4 illustrates a display of a home page for a user of a secure document exchange portal. The home page allows for the user to make requests for information, case search, documents, or invoicing. Access to each category may be limited from one user to another by way of authorization mechanisms as described herein.

### 2. Automatic Document Classification Tool for Large Electronic Files

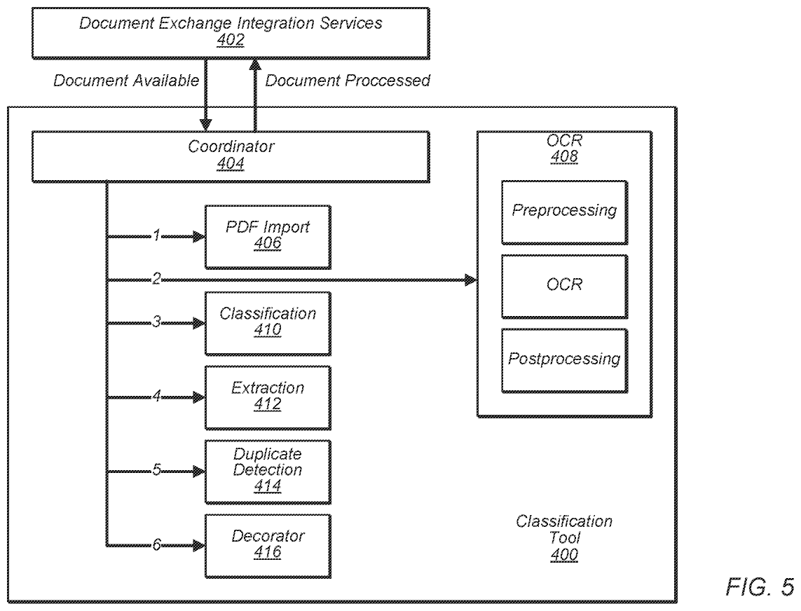

FIG. 5 illustrates a classification tool according to one embodiment. Classification tool 400 may process documents received from document exchange integration services 402. Document exchange integration services may provide a notification that a document is available for classification. Coordinator 404 may receive each document and implement operations in classification tool 400. Processing may include PDF import 406, OCR 408, classification 410, extraction 412, duplicate detection 414, and insertion of bookmarks by decorator 416. In one embodiment, a classification tool includes an import tool, OCR, classification, date extraction, duplicate detection, decorator and document Q/A.

The duplicate detection tool may find duplicate information within a document or a case file. For example, a file may contain multiple copies of a particular progress report that were uploaded by different stakeholders at different times. Finding duplicates may involve searching the hash keys vs. comparing the text of each page. In one embodiment, duplicate detection leverages a Locally Sensitive Hash (LSH). The tool can also be used to find similar pages. With a 64-bit hash each bit represents approximately 1.56% of the page, so difference can be computed by the number of differing bits.

Information extraction tool supports extraction of attributes for downstream processing. In one embodiment, information extraction is used to extract service dates allowing downstream processes to provide a timeline of events.

Corrections to classification and extraction errors can be captured with a QA Tool prior to releasing documents to physicians. Corrections captured by the QA Tool may also be used to improve classification and extraction accuracy.

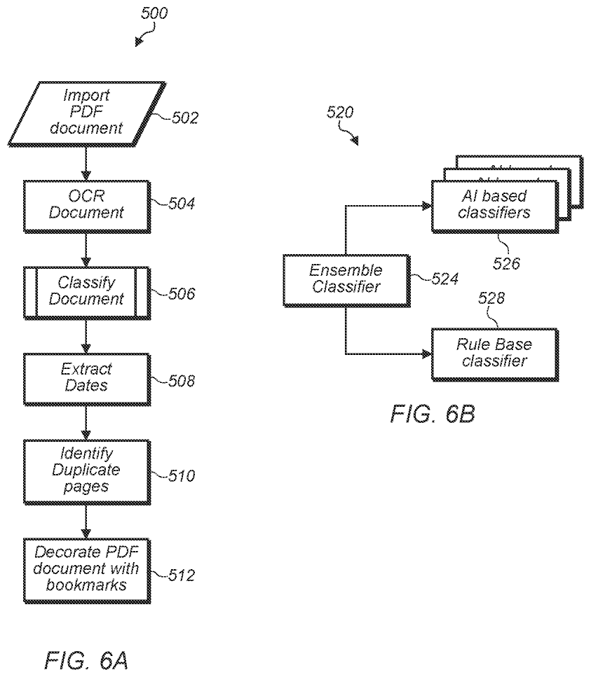

FIG. 6A illustrates one embodiment of a flowchart illustrating a document classification flow (500). In some embodiments of the method, files are imported (502). The imported file may undergo OCR (504) to extract text from scanned pages. Documents within the imported file may be classified (506) using a classification tool. In some embodiments, the method may include extracting specific information and metadata from the imported documents. The specific information may include dates (e.g., such that the documents may be organized according to their creation and/or entry date) and other meta-data. The method may include identifying (510) duplicate information and specifically duplicate pages within the imported documents using a duplicate detection tool. The duplicate detection tool may find duplicate information within a document or a case file. For example, a file may contain multiple copies of a particular progress report that were uploaded by different stakeholders at different times. The method may include adding (512) bookmarks with a decorator tool. The bookmarks may provide quick access to identified information. The metadata from this pipeline may also be used by the case viewer to facilitate a case-level view.

FIG. 6B illustrates one embodiment of a flowchart illustrating an ensemble classifier tool (524). The ensemble classifier tool may be applied to identify specify information in the documents. The ensemble classifier tool may include different classifiers such as AI based classifiers (526) and rule base classifiers (528).

In some embodiments, the classification tool may classify a portion of the file as a particular type of document. Examples of document types into which portions of the file may be classified include: a request for authorization, a utilization review determination, or a physician's progress report. In some embodiments, the classification tool may function to reduce redundancies and/or irrelevant data within the file. For example, the classification tool may identify duplicate information (e.g., duplicate information may be submitted from multiple sources).

In some embodiments, the classifiers may include an ensemble approach that combines the predictions from multiple classifiers for a final prediction. The classification framework allows the configuration of different classifiers, such as AI based classifiers, and classifier proxies such as thresholding classifiers, weighting classifiers. The ensemble can use different mechanisms for a final prediction such as weighting.

The classification framework also includes tools to measure the accuracy of the classifiers and to retrain the classifiers as needed.

In some embodiments, the classifiers may include rules-based classifiers. In some cases, a rules-based classifier identifies phrases and associates them with specific labels. An example of a rule-based system is the domain-specific expert system that uses rules to make deductions or choices. For example, an expert system might help a doctor choose the correct diagnosis based on a cluster of symptoms. Rule-based systems may be used to perform lexical analysis to compile or interpret computer programs, or in natural language processing.

A rule-based system may include a rule base and an inference engine or semantic reasoner. A rule base may include a list of rules. An inference engine may infer information or take action based on the interaction between input data and the rule base.

In some embodiments, various other artificial intelligence classifiers, such as maximum entropy, may be used in addition to or instead of the rules-based classifiers. Maximum entropy may be referred to as multinomial logistic regression. Multinomial logistic regression is a classification method that generalizes logistic regression to multiclass problems, or a model that is used to predict the probabilities of the different possible outcomes of a categorically distributed dependent variable, given a set of independent variables. Multinomial logistic regression may be used when the dependent variable in question is nominal and for which there are more than two categories. Rule-based systems and maximum entropy systems may be used in combination with one another (e.g., comparing the scores from the results to determine the best result). In some embodiments, search tools may rely on visual or non-text indicators, such that images themselves are searchable (which may be useful considering the number of medical images which may associated with a case).

In some embodiments, based on the output of the classifiers, scores are computed for various portions of the file. For each portion, the best score may be determined and used as an outcome for a particular portion of the file. In some embodiments, the presence of certain phrases may be used to identify a portion of the file as a particular type of information, such as a physician's progress report.

In some embodiments, a method may include assigning at least one type of bookmark based upon the classification. Based on the classifications, the file is decorated with bookmarks that match the determined classifications. In some embodiments, dates or times are determined for some or all of the classified information. The document classifications, dates, bookmarks, and other information extracted or determined during classification may be sorted, arranged, and presented to a user (e.g., a physician reviewing the file) to facilitate the user's review of the file. In some embodiments, the user may be presented with a dashboard that allows the user to navigate through the bookmarks in a particular case, by document class type, date, and/or other user-selected criteria.

In some embodiments, classifying elements of information in a file includes refining the automated classification method based upon manual corrections of the classification method. In some embodiments, a quality assurance tool 620 is used to assess the output of the document classification tool 610. The quality assurance tool may be operated by a quality assurance (QA) reviewer 630 (e.g., a clerk or nurse) to review the classification output and assess the quality of the classification output.

In some embodiments, the QA reviewer 630 manually identifies any errors or omissions in the classification output (e.g., a document identified with the wrong document type identifier). The QA reviewer may use hot keys or similar mechanisms to quickly make additions or changes to the file. In cases where there are errors, the QA reviewer may fix errors. For example, the QA reviewer may make corrections, add a bookmark, remove a bookmark, and/or change a bookmark.

In some embodiments, results of the review may be used to automatically train the document classification tool. For example, the system may use the information from the QA review to automatically modify the classifier to more accurately identify or date information in future use of the classification tool.

In certain cases, the classification tool can assess graphical content in addition to, or in conjunction with, textual information in a file to classify portions of the file.

### 3. Case-Level Review Tool for Physicians

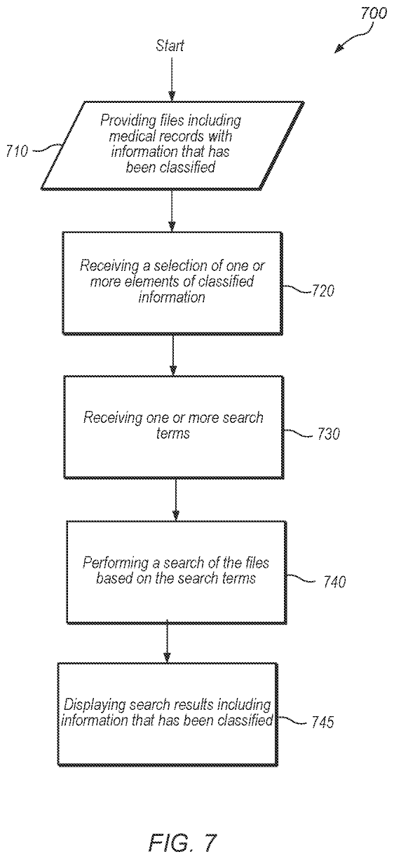

FIG. 7 illustrates one embodiment of a flowchart illustrating a method (700) of assisting physicians using a physician's search tool 810 based on information that has been automatically classified. In some embodiments, the method may include providing (710) one or more files comprising scanned medical records. At least one of the files may include information classified as pre-defined information types. In some embodiments, the method may include receiving (720), from a physician user, a selection of one or more elements of classified information. One or more search terms may be received from the physician user (730). The physician search tool may perform one or more searches based on the search terms. (740). Results of the search, including elements of classified information, may be displayed to the physician user (745).

In some embodiments, the files include two or more files associated with the same medical claim. In some embodiments, the method may include searching the content of the files. In some embodiments, the method may include creating a summary of a content of the at least one of the files.

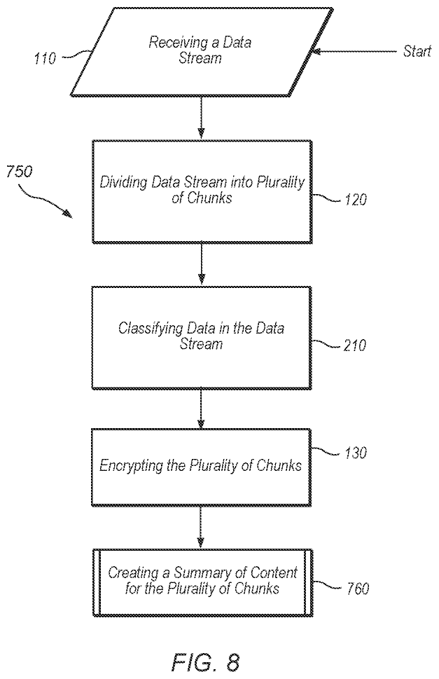

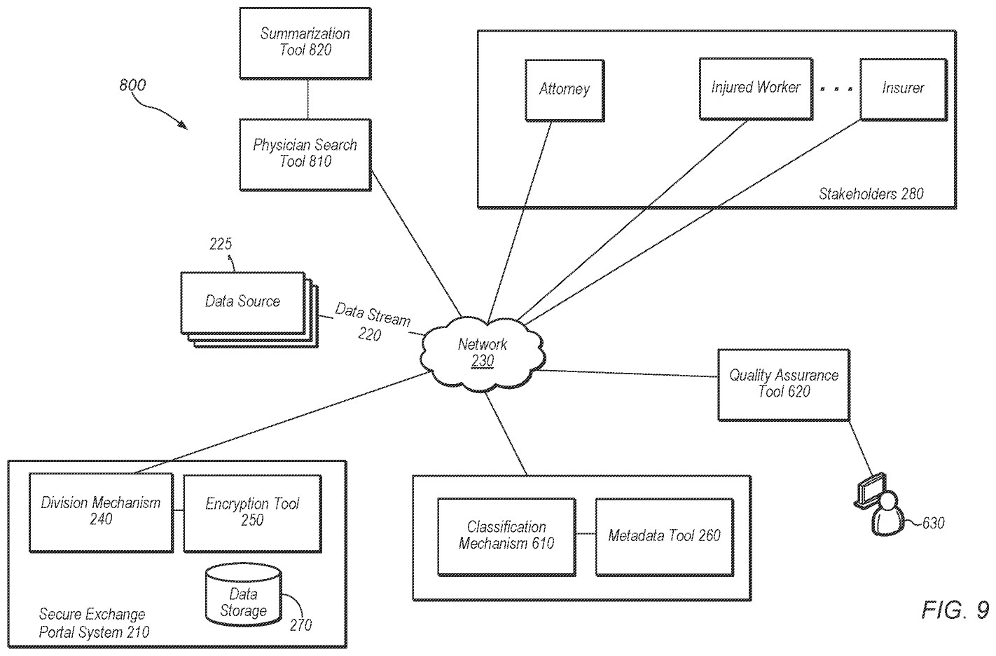

FIGS. 8 and 9 illustrate one embodiment of a flowchart illustrating a method (750) and 800 of assisting physicians using a physician's search tool 810 based upon automatically classifying information using a secure exchange portal system 210 for independent medical review. In some embodiments, a method as described herein may include assisting physicians using a physician's search tool based upon automatically classifying information using a secure exchange portal system (e.g., for independent medical review). A review tool may enable a physician to review, search, and annotate large electronic files that contain all of the records for a case. As discussed herein, the file may include, for example, an image pdf file produced by scanning numerous paper documents. The case may be, for example, an independent medical review of a worker's compensation claim. In some embodiments, the method may be as described previously herein (e.g., receiving 110, dividing 120, encrypting 130, classifying 506, etc.) and the method further including creating (760) a summary of content for the plurality of chunks based upon the classification and/or the metadata using a summarization tool 820.

In some embodiments, a physician may view a case summary page for any of the cases assigned to the physician. The case summary page may include information on the contents of the electronic files(s) for the case. In some embodiments, the information may include a list of documents contained in the file(s). The document listing may be generated using an automatic document classification tool that has been applied to the contents of the files as described herein previously. In some embodiments, a document classification may be run on the text (e.g., OCR text) of one or more electronic files or scanned paper documents. In some embodiments, bookmarks may be added automatically corresponding to each document found based on the results of the classifier.

In some embodiments, the review tool allows for insertion of notes and case alerts by personnel that review the file prior to physician review (e.g., a nurse). For example, a nurse may add a case alert that includes a key fact about the case or that the case needs to be expedited. There may be different types of notes available for insertion. For example, notes may be color coded according to different predetermined uses and/or according to a user's preferences. In some embodiments, notes may include different priority levels (e.g., by color). For example, an inserted note including a specific priority level may automatically self-indicate (e.g., begin flashing, overlay all existing open windows, etc.) when a particular file the note is associated with is accessed.

In some embodiments, on a summary page for a case, the document listing may be arranged to facilitate quick and efficient review by the user (e.g., a physician). Examples of document types may include, but are not limited to: a request for authorization, a utilization review determination, or a physician's progress report. In some embodiments, the document listing may be arranged by type, then by document, then by date, then by page. Bookmarks, tags for notes, and/or case description may be displayed as desired by a user.

In some embodiments, a search mechanism in the review tool allows the user (e.g., physician) to search contents of the files. In some embodiments, the physician uses keywords and phrases to conduct the search. In some embodiments, the search mechanism employs fuzzy searching. Fuzzy searching allows the physician to search for contents using imprecise search criteria (where, for example, a precise search is not possible or may not find all of the relevant content). Approximate string matching (fuzzy string searching) may include finding strings that match a pattern approximately (rather than exactly). Approximate string matching may include finding approximate substring matches inside a given string and finding dictionary strings that match the pattern approximately. The closeness of a match may be measured, for example, in terms of the number of primitive operations necessary to convert the string into an exact match. In some embodiments, a search mechanism allows proximity searches. A proximity search may include a search for information where two or more separately matching term occurrences are within a specified distance. Distance may be measured by the number of intermediate words or characters. In some embodiments, some implementations of a proximity search may impose a constraint on the word order. In some embodiments, the search mechanism may account for aliases or synonyms (e.g., scientific name of a drug being associated with a brand name for the same drug). In some embodiments, search results may be in the form of line snippets including the search term, or highlighting applied directly onto the file image at the location of the search result.

In some embodiments, upon completion of the physician's review, the physician may write the physician's report directly to a backend system using the review tool. The review tool may include an editor that can be used by the physician to enter the report.

In some embodiments, using the review tool, a physician can access specific information or portions of the records for the case without downloading or unencrypting the entire contents of the electronic files for a case. This avoids the need for a physician to transfer and store large sensitive files on the physician's computing device. In some embodiments, the files for the case are indexed for content, such as particular drug names, medical conditions, or other relevant information.

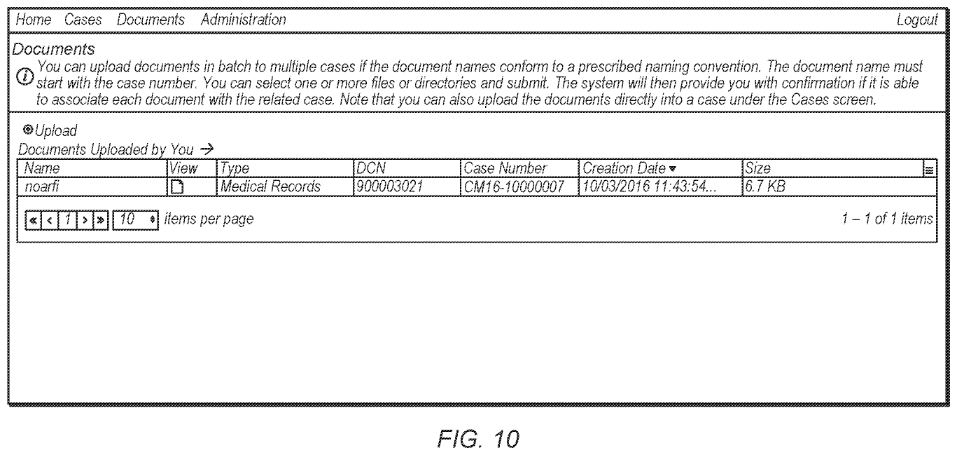

FIG. 10 illustrates a case document management display for a physician search tool according to one embodiment.

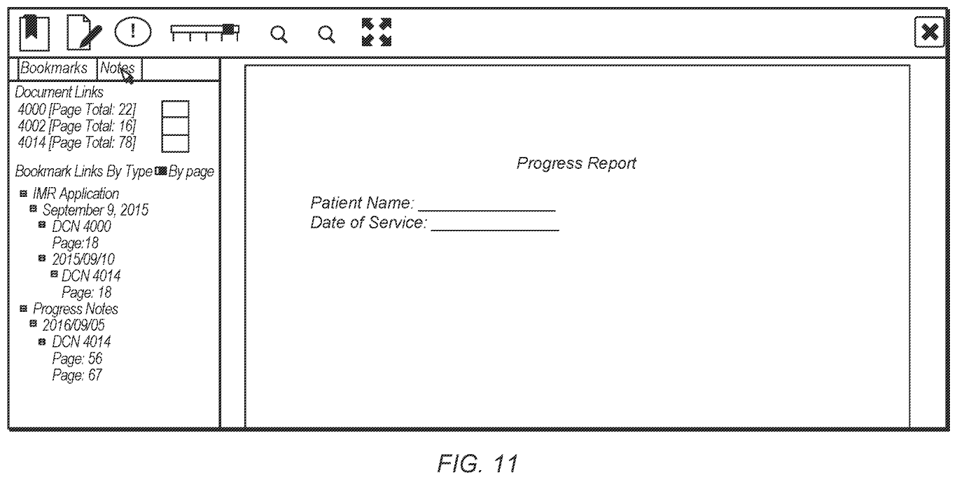

FIG. 11 illustrates a display of physician search tool with a Bookmarks tab selected and bookmark links arranged by type.

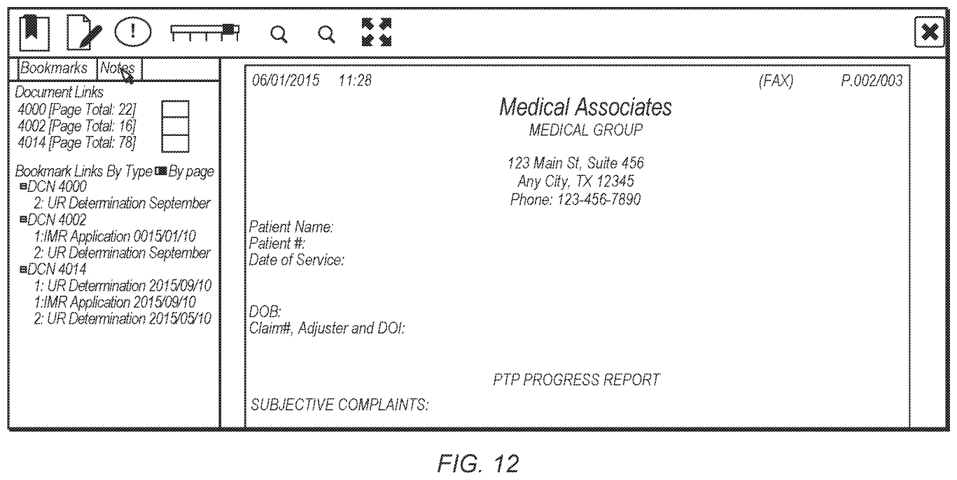

FIG. 12 illustrates a display of a physician search tool with a Bookmarks tab selected and bookmark links arranged by page.

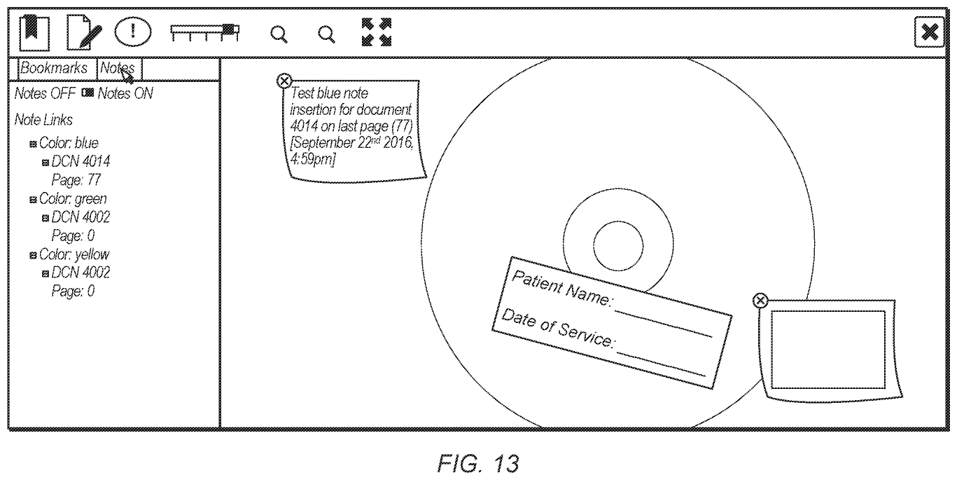

FIG. 13 illustrates a display of a physician search tool with a Notes tab selected and stick notes attached to a displayed document.

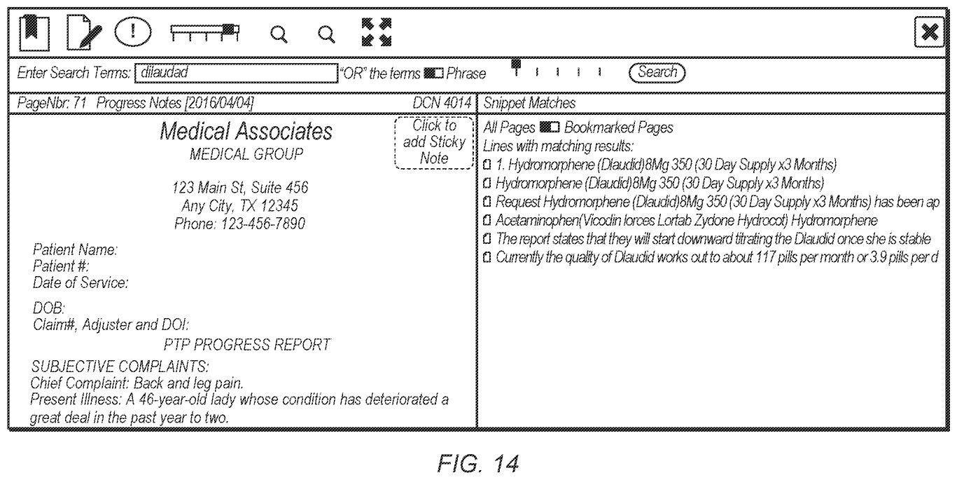

FIG. 14 illustrates a display of a physician search tool with line-by-line search results shown next to a document classified by the system as a Progress Note.

The memory medium may store a software program or programs operable to implement embodiments as described herein. The software program(s) may be implemented in various ways. A CPU executing code and data from the memory medium may include a means for creating and executing the software program or programs according to the embodiments described herein.

Although various embodiments above describe methods and processes used to process information in an independent medical review in a case under appeal in a worker's compensation case, systems and processes described herein (including the secure document exchange, classification, and search tools) may be used in other worker's compensation claims, medical claims, or other processes involving review of information contained in large files.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as embodiments. Elements and materials may be substituted for those illustrated and described herein, parts and processes may be reversed, and certain features of the invention may be utilized independently, all as would be apparent to one skilled in the art after having the benefit of this description of the invention. Methods may be implemented manually, in software, in hardware, or a combination thereof. The order of any method may be changed, and various elements may be added, reordered, combined, omitted, modified, etc. Changes may be made in the elements described herein without departing from the spirit and scope of the invention as described in the following claims.

1-68. (canceled)

69. A method, comprising:
receiving, by a computer system, a data stream from at least a first source, wherein the data stream includes a plurality of individual documents having data for a medical claim being evaluated by an independent medical review;
dividing, by the computer system, the received data stream into a plurality of sets of documents, wherein each set of documents includes a different portion of the documents from the data stream, the different portions being divided at transitions between the individual documents, and wherein a first set of documents includes multiple individual documents;
after dividing the received data stream into the plurality of sets, applying a classification tool to the first set of documents to classify the first set of documents, wherein the classification tool automatically classifies the first set of documents by:
identifying textual medical data and metadata in the individual documents in the first set of documents, wherein the textual medical data includes at least one medical identifier in the individual documents; and
determining a type of document for the individual documents in the first set of documents based on the identified textual medical data and the identified metadata;
encrypting the first set of documents, wherein encrypting the first set of documents includes generating one or more encryption keys for the first set of documents;
generating, by the computer system, metadata for the encrypted first set of documents, wherein the generated metadata is generated based, at least in part, on the type of documents for the individual documents in the encrypted first set of documents determined by the classification tool and the encryption keys for the encrypted first set of documents; and
transmitting the encrypted first set of documents and the generated metadata to a stakeholder associated with the independent medical review in response to a request by the stakeholder.

70. The method of claim 69, wherein the generated metadata includes the encryption keys.

71. The method of claim 70, wherein the encryption keys are implemented to restrict access to the first set of documents to the stakeholder.

72. The method of claim 69, wherein the generated metadata includes coded medical identifiers.

73. The method of claim 69, wherein the identified metadata includes coded medical identifiers.

74. The method of claim 69, wherein the identified metadata includes document metadata and medical case metadata.

75. The method of claim 74, wherein the document metadata includes one or more of the following data: document unique identifier, document creation date, document last update date, document source, and document type.

76. The method of claim 74, wherein the medical case metadata includes one or more of the following data: medical case identifier, medical case status, medical case status date, and medical case last update date.

77. The method of claim 69, wherein generating the metadata includes generating a first set of metadata and a second set of metadata, wherein the first set of metadata is implemented to restrict access to a first portion of the first set of documents to a first group and a second set of metadata is implemented to restrict access to a second portion of the first set of documents to a second group, wherein the first group is different from the second group.

78. The method of claim 69, wherein the classification tool automatically classifies the first set of documents by additionally:
identifying graphical content in the individual documents in the first set of documents; and
determining the type of document for the individual documents in the first set of documents based on the identified textual medical data, the identified metadata, and the identified graphical content.

79. The method of claim 69, wherein the medical claim being evaluated includes a worker's compensation claim.

80. The method of claim 79, further comprising receiving, by the computer system from the stakeholder, a resolution to the worker's compensation claim.

81. The method of claim 69, further comprising generating, by the computer system, a summary that includes a list of data in the first set of documents, wherein the list of data is arranged according to the types of documents in the first set of documents determined by the classification tool.

82. The method of claim 81, wherein the summary is generated as part of the generated metadata.

83. The method of claim 81, wherein the types of documents include a request for authorization, a utilization review determination, or a physician's progress report.

84. A non-transitory computer-readable medium having program instructions stored thereon that are executable by a computer system to perform operations comprising:
receiving a data stream from at least a first source, wherein the data stream includes a plurality of individual documents having data for a medical claim being evaluated by an independent medical review;
dividing the received data stream into a plurality of sets of documents, wherein each set of documents includes a different portion of the documents from the data stream, the different portions being divided at transitions between the individual documents, and wherein a first set of documents includes multiple individual documents:
after dividing the received data stream into the plurality of sets, applying a classification tool to the first set of documents to classify the first set of documents, wherein the classification tool automatically classifies the first set of documents by:
identifying textual medical data and metadata in the individual documents in the first set of documents, wherein the textual medical data includes at least one medical identifier in the individual documents; and
determining a type of document for the individual documents in the first set of documents based on the identified textual medical data and the identified metadata;
encrypting the first set of documents, wherein encrypting the first set of documents includes generating one or more encryption keys for the first set of documents;
generating metadata for the encrypted first set of documents, wherein the generated metadata is generated based, at least in part, on the type of documents for the individual documents in the encrypted first set of documents determined by the classification tool and the encryption keys for the encrypted first set of documents; and
transmitting the encrypted first set of documents and the generated metadata to a stakeholder associated with the independent medical review in response to a request by the stakeholder.

85. The non-transitory computer-readable medium of claim 84, wherein the generated metadata includes the encryption keys and coded medical identifiers.

86. The non-transitory computer-readable medium of claim 84, wherein the identified metadata includes document metadata and medical case metadata, the medical case metadata including coded medical identifiers.

87. The non-transitory computer-readable medium of claim 86, wherein the document metadata includes data for document unique identifiers, document creation dates, document last update dates, document sources, and document types, and wherein the medical case metadata includes data for medical case identifiers, medical case status, medical case status dates, and medical case last update dates.

88. The non-transitory computer-readable medium of claim 84, further comprising receiving, from the stakeholder, an evaluation result from the independent medical review.
